(19) United States
(12) Patent Application Publication
(10) Pub. No.: US 2025/0099246 A1
(43) Pub. Date: Mar. 27, 2025

(54) ADJUSTABLE AORTIC ANNULOPLASTY RINGS FOR SYMMETRIC AORTIC ANNULUS REDUCTION AND METHODS FOR USE

(71) Applicant: The Board of Trustees of the Leland Stanford Junior University, Stanford, CA (US)

(72) Inventors: Matthew Park, Piedmont, CA (US); Yuanjia Zhu, Stanford, CA (US); Y. Joseph Woo, Stanford, CA (US)

(21) Appl. No.: 18/967,557

(22) Filed: Dec. 3, 2024

Related U.S. Application Data
(63) Continuation of application No. PCT/US2023/024387, filed on Jun. 4, 2023.
(60) Provisional application No. 63/349,068, filed on Jun. 4, 2022.

(51) Int. Cl. A61F 2/24

(52) U.S. Cl. CPC A61F 2/2445; A61F 2220/0075; A61F 2220/0091; A61F 2230/0006; A61F 2250/001

(57) ABSTRACT

Adjustable aortic annuloplasty rings are disclosed for constricting a valve annulus, comprising an annular body sized for positioning around an exterior of the valve annulus, the annular body comprising a compliant uni-body driven by a Bowden cable constriction mechanism to adjust an inner diameter of the device.

### RELATED APPLICATION DATA

The present application is a continuation of co-pending International Application No. PCT/US2023/024387, filed Jun. 4, 2023, which claims benefit of U.S. provisional application Ser. No. 63/349,068, filed Jun. 4, 2022, the entire disclosures of which are expressly incorporated by reference herein.

### STATEMENT REGARDING FEDERALLY SPONSORED RESEARCH AND DEVELOPMENT

None.

### TECHNICAL FIELD

The present application relates to medical devices and, more particularly, to annuloplasty devices and to methods for making and using such devices.

### BACKGROUND

Valve-sparing aortic root replacement (“VSARR”) is a procedure developed to preserve the aortic valve (“AV”) apparatus while replacing an aneurysmal aortic root with a synthetic graft. The procedure may be used for patients experiencing aortic valve regurgitation (“AR”) due to annular dilation. This type I AR is largely due to geometric disturbances to the functional aortic annulus, such as an enlarged aortic root, that result in inadequate leaflet coaptation. Depending on the location of root dilation, VSARR may be an effective procedure to treat type I AR with excellent outcomes. In general, the VSARR procedure can be performed using either the reimplantation or remodeling techniques, the latter of which may also require use of an annuloplasty ring and may be unable to correct the dilated aortic annulus, e.g., often observed in young patients with connective tissue diseases, which can lead to repair failure with recurrent AR. However, even while using the reimplantation technique, residual AR can occur in patients.

Aortic annulus stabilization was first proposed several decades ago. Different aortic annuloplasty techniques and devices have been proposed to stabilize the annulus and eliminate residual AR, further improving outcomes, such as external polytetrafluoroethylene (PTFE) suture technique, flexible annuloplasty ring implantation to eliminate ventriculo-aortic junction dilation, sub-commissural annuloplasty, the single PTFE suture technique, and implantation of a rigid, internal annuloplasty ring. However, these techniques can be technically challenging to perform, and the use of such devices can significantly increase cardiopulmonary bypass and aortic cross-clamp times.

Therefore, there is a need for improved devices for stabilizing the aortic valve annulus and/or reducing aortic regurgitation.

### SUMMARY

The present application is directed to medical devices and, more particularly, to annuloplasty devices and to methods for making and using such devices.

For example, provided herein is a novel annuloplasty device that allows for a simple and easily repeatable solution for off-pump adjustment of AV annular diameter for precise, patient-specific optimization of valve coaptation, hemodynamics, and performance, e.g., including a uni-body, 3D-printed, compliant, external, adjustable, symmetrically extensible (EASE) aortic annuloplasty ring.

In one example, a surgically implantable prosthetic device is provided that is configured to be implanted externally to an aortic valve annulus, e.g., above the coronary arteries but in line with the commissures and nadirs of the existing valve. The device may include a flexible biocompatible material designed in a way such that a tightening mechanism (e.g., an embedded suture or other filament) may be used to reduce the diameter of the device, e.g., by symmetrically reducing the distances between the commissures and nadirs of the valve.

In accordance with one example, a prosthetic annuloplasty ring is provided for constricting a valve annulus that includes an annular body sized for positioning around an exterior of the valve annulus, the annular body comprising a compliant uni-body driven by a Bowden cable constriction mechanism to adjust an inner diameter of the device.

In accordance with another example, a prosthetic annuloplasty device is provided for constricting a valve annulus that includes an annular body defining a perimeter sized for positioning around an exterior of the valve annulus; a plurality of channel elements on the annular body spaced apart from one another around the perimeter; and a filament slidably received within channels in the channel elements around the perimeter for adjusting an inner diameter of the annular body by tightening the filament. In one example, the annular body may include a plurality of first elements and a plurality of second hinge elements alternating around the perimeter. The channel elements, carried on the first elements and/or the channel elements, may be arranged on respective elements such that the channels are aligned generally circumferentially around the perimeter.

In accordance with yet another example, a prosthetic annuloplasty device is provided for constricting a valve annulus that includes an open annular or “C shaped body defining a perimeter sized for positioning around an exterior of the valve annulus and including opposite ends; a plurality of channel elements on the annular body spaced apart from one another around the perimeter; and a filament slidably received within channels in the channel elements and extending around the perimeter for adjusting an inner diameter of the annular body by tightening the filament.

In accordance with still another example, a method is provided for constricting a valve annulus that includes positioning an annular body externally around the valve annulus; introducing an elongate filament through a plurality of channels spaced apart from one another around a perimeter of the annular body; and tightening the filament to reduce the perimeter of the annular body to constrict the valve annulus.

Other aspects and features of the present invention will become apparent from consideration of the following description taken in conjunction with the accompanying drawings.

### BRIEF DESCRIPTION OF THE DRAWINGS

It is believed the present invention will be better understood from the following description of certain examples taken in conjunction with the accompanying drawings, in which like reference numerals identify the same elements and in which:

FIGS. 1A and 1B show an example of an annuloplasty ring that includes an annular body including a plurality of channels spaced apart around an outer perimeter of the body for receiving a suture or other filament for adjusting a size of the annular body.

FIGS. 1C and 1D show the annuloplasty ring of FIGS. 1A and 1B with a suture received in the channels to adjust the size of the annular body, e.g., from a first diameter shown in FIG. 1C to a smaller second diameter shown in FIG. 1D.

FIGS. 2A and 2B show an exemplary annuloplasty ring being positioned around an enlarged aortic root (FIG. 2A) and tightened to constrict the aortic root (FIG. 2B).

FIG. 3 shows another example of an annuloplasty ring including a “C” shaped body including a suture slidably received in channels around the perimeter of the body.

FIGS. 4A-4D show an exemplary annuloplasty ring, such as that shown in FIGS. 1A-1D, mounted to a model of an aortic valve annulus. In particular, FIGS. 4C and 4D show the annuloplasty ring being adjusted using a suture to constrict the valve annulus.

FIGS. 5A-5C are exemplary images of en face views of an aortic valve in an ex vivo left heart simulator.

FIGS. 6A and 6B are graphs showing exemplary mean aortic flow and pressure tracings of baseline, valve-sparing aortic root replacement (VSARR) model, and post-aortic ring annuloplasty repair.

The drawings are not intended to be limiting in any way, and it is contemplated that various examples of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

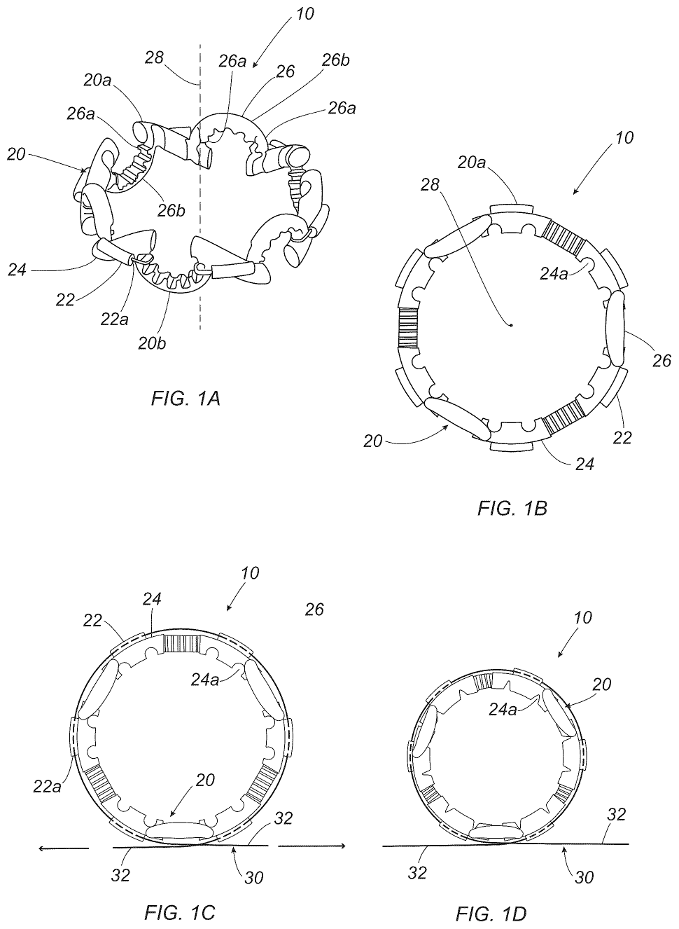

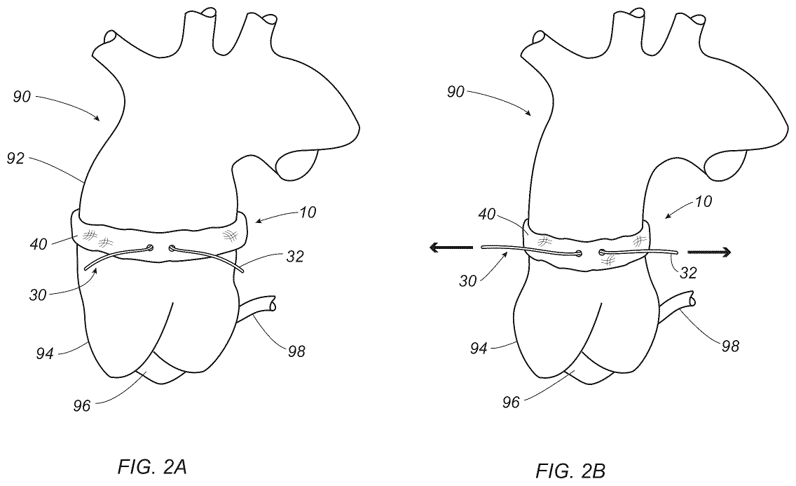

Turning to the drawings, FIGS. 1A-1D show an example of an adjustable aortic annuloplasty device 10 including an enclosed annular ring or body 20 and a suture or other filament 30, which may be configured to provide substantially symmetric, circular reduction of a valve annulus, e.g., while avoiding tissue pinching and/or asymmetric rotation of the annulus. Generally, the device 10 may be configured to be implanted externally around a valve annulus, e.g., around an enlarged region of an aortic valve annulus as shown in FIGS. 2A and 2B, such that the device 10 may be used to reduce the diameter of the valve annulus via symmetric radial constriction, similar to radial clamping, as opposed to circumferential reduction, such as suture tightening, in order to maintain the quasi-symmetric aortic root anatomy. Although the device 10 may have particular use for constricting an aortic valve annulus, it will be appreciated that the devices and methods may be used in other locations where it is desired to surround and constrict an enlarged anatomical structure.

In the example shown, the device 10 may include a compliant, uni-body driven by a Bowden cable constriction mechanism, implemented with the suture 30. In an exemplary method, the complete annuloplasty ring may be designed in an aortic annulus geometry and manufactured using a semi-flexible, biocompatible, 3D-printed polyurethane material, as described further elsewhere herein.

The annular body 20 may include one or more features, e.g., including precise kerfing in the geometry integrally formed in the body during manufacturing, to control the dynamic compliance mechanism for adjusting the inner diameter of the device 10, e.g., as shown in FIGS. 1C and 1D. As best seen in FIGS. 1A and 1B, the annular body 20 includes a plurality of channel elements 22 spaced apart from one another around the perimeter of the annular body 20 for slidably receiving the suture 30. For example, the channel elements 22 may be integrally formed or attached to substantially straight or first elements 24 and/or hinged or curved second elements 26. The second elements 26 may be provided between the first elements 24, e.g., such that the first and second elements 24, 26 alternate around the perimeter of the annular body 20, or are otherwise interconnected to provide an enclosed annular body 20. With this mechanism, the annular body 20 may allow for constriction substantially symmetrically to adjust the inner diameter of the device 10 upon tightening down a suture 30 received in channels 22a of the channel elements 22.

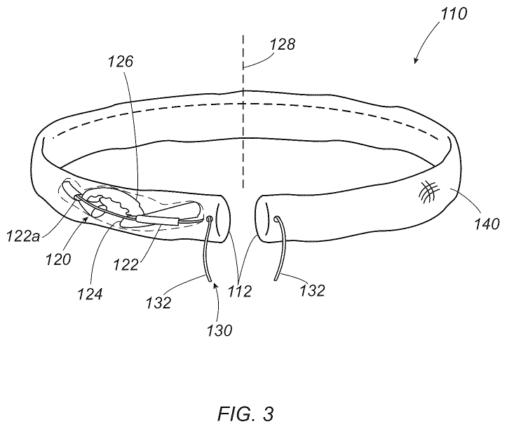

Alternatively, as shown in FIG. 3, an annuloplasty device 110 including a “C” shaped or other open or incomplete annular ring or body 120 may be provided that does not entirely enclose the perimeter, e.g., by omitting one of the hinge elements 26. In this alternative, the unconnected ends 112 of the body 120 may be separated to accommodate positioning the device 110 around a valve annulus and then positioned adjacent one another, rather than positioning an enclosed annular body around the valve annulus, as described further elsewhere herein. In this alternative, the suture 130 (or other filament) may be inserted circumferentially through channels 122a during assembly of the device 110 such that ends 132 of the suture 130 are disposed adjacent respective ends 112 of the body 120, e.g., extending through openings in a fabric covering 140 provided on the device 120. Alternatively, the suture 130 may be threaded or otherwise inserted through the channel elements 122 after positioning the device 110 around the valve annulus and tightened, as desired, to reduce the diameter of the valve annulus, as described further elsewhere herein.

Returning to FIGS. 1A and 1B, the annular body 20 may surround a central axis 28 of the device 10, e.g., such that an upper end 20a and a lower end 20b of the annular body 20 are aligned generally opposite one another along the axis 28. In the example shown, the first elements 24 may be oriented circumferentially around the perimeter of the annular body 20 yet extending diagonally relative to a plane orthogonal to the axis 28. The channel elements 22 may be arranged on the outer perimeter of respective first elements 24 such that the channels 22a are aligned circumferentially around the body 20, e.g., such that the channels 22a lie generally with a plane orthogonal to the axis 28. Thus, in this arrangement, the channel elements 22 may be attached diagonally relative to a length of the respective first elements 24 to align the channels 22a around the circumference of the body 20.

The hinges second elements 26 may define a generally “C” or other arcuate shape with ends 26a that are coupled to the adjacent first elements 24. Consequently, when the suture 30 is tightened, an intermediate region 26b of the hinge elements 26, i.e., between the ends 26a, may bend or otherwise deform, e.g., to direct the ends 26a towards one another around the circumference, to reduce the diameter of the annular body 20. In the example shown, the axial orientation of the hinge elements 26 may alternate around the perimeter of the annular body 20 between adjacent first elements 24. For example the ends 26a of a first hinge element 26 may be located adjacent the upper end 22a and the intermediate region 26b of the first hinge element 26 located adjacent the lower end 22b, and the ends 26a of the adjacent hinge elements 26 may be located adjacent the lower end 22a and the intermediate region 26b of the adjacent hinge elements 26 may be located adjacent the upper end 22a, which may more evenly distribute forces as the annular body 20 is constricted.

Optionally, as best seen in FIG. 1B, the first elements 24 may include one or more recesses or other kerfing features 24a, e.g., located along the inner perimeter of the annular body 20, which may accommodate bending of the first elements 24 around the perimeter, which may facilitate radial constriction of the annular body 20 and/or minimize tortional forces being applied to tissue of the valve annulus as the annulus is constricted. For example, each first element 24 may include a slot or groove 24a adjacent each end of the channel element 22, e.g., extending along the first element 24 substantially parallel to the axis 28, to provide hinges around the circumference of the annular body 20, which may be compressed or otherwise deformed when the device 10 is compressed, e.g., as shown in FIG. 1D.

Optionally, the device 10 may include one or more additional features. For example, the annular body 20 may be covered with Dacron or other fabric 40, e.g., as shown in FIGS. 2A and 2B, to facilitate securing the device 10 and/or encouraging tissue ingrowth. The ends 32 of the suture 30 may exit the fabric 40 adjacent one another, e.g., through a single opening or separate openings in the fabric 40. In addition or alternatively, the device 10 may be incorporated or attached to a Dacron graft (not shown), e.g., such that a single graft device may be provided that may be implanted together, e.g., during aortic root surgery.

The device 10 may be formed using a variety of materials and methods. For example, the annular body 20 may be formed by 3D printing, molding, casting, machining, and the like, such that the elements 22-26 of the annular body 20 are integrally formed together. Alternatively, one or more of the elements 22-26 may be formed separately and attached together, e.g., by one or more of bonding with adhesive, fusing, sonic welding, and the like, to provide the final annular body 20.

The annular body 20 may be formed from biocompatible materials, e.g., having a conformability to accommodate constriction of the device 10, e.g., semi-flexible polyurethane (FPU50, Carbon, Redwood City, CA) or other plastic, metal, or composite material. The annular body 20 may be biased to a relaxed initial size and/or shape, e.g., the size shown in FIG. 1C, but may be radially compressible by tightening the suture 30, e.g., with one or both of the first elements 24 and hinge elements 26 bending or otherwise deforming to reduce the inner diameter of the annular body 20, e.g., as shown in FIG. 1D. If the suture 30 is released, the annular body 20 may resiliently return towards the relaxed size and/or shape. Alternatively, the material of the annular body 20 may be malleable, e.g., such that the annular body 20 may be compressible by tightening the suture 30 but may at least partially retain the compressed size and/or shape, e.g., if tension from the suture 30 is released.

Alternatively, as shown in FIG. 3, an open or incomplete ring device 110 may be provided, e.g., similar to the annular body 20, except with a discontinuity, e.g., to provide a “C” or other shaped body 120 including two free ends 112 adjacent one another. For example, one or more of the elements 24, 26 may be omitted from the annular body 120 adjacent the ends 112 to provide an open ring. A suture or other filament 130 may be introduced through the passages 122a around the perimeter of the body 120 and similarly tightened to compress the body 120. The free ends 112 may be configured to abut one another during compression and/or, optionally, may include one or more connectors (not shown) to couple the ends 112 together during implantation, to allow subsequent tightening of the suture 130. Alternatively, the ends 112 may remain separated when the device 110 is tightened, and/or, optionally may be sutured or otherwise secured to the valve annulus during implantation, e.g., before or after tightening.

Turning to FIGS. 2A and 2B, during use, the annuloplasty device 10 may be implanted externally around a valve annulus, e.g., an aortic valve annulus, to stabilize the annulus and/or reduce regurgitation. For example, as shown in FIG. 2A, an aorta 90 is shown that includes an enlarged aortic root 92 above the sinus of Valsalva 93 adjacent the aortic valve 96. Initially, the annular body 20 may be positioned externally around the aorta 90, e.g., around the enlarged region 92. In one approach, the device 10 may be positioned over a severed end of the aorta (not shown) and positioned around the enlarged region 92 when the aorta 90 is open and the aortic valve 96 is accessible, e.g., after aortic valve repair or a VSARR procedure.

Once the annular body 20 is positioned at a desired location, e.g., above the coronary arteries 98 but in line with the commissures and nadirs of an existing aortic valve 96, the ends 32 of the suture 30 may be pulled to tighten the device 10 around the enlarged region 92, e.g., as shown in FIG. 2B. Optionally, during assembly of the device 10 (e.g., during manufacturing or immediately before implantation), the suture 30 may be introduced through the passages 22a such that the ends 32 exit the fabric covering 40, e.g., with sufficient slack to prevent the annular body 20 from being subjected to undesired compression during introduction and positioning. Alternatively, the device 10 may be introduced without the suture 30, and the suture 30 may be introduced through the passages 22a around the perimeter after positioning around the enlarged region 92, and then tightened to reduce the perimeter of the annular body to constrict the valve annulus.

Once the suture 30 is tightened to constrict the aortic root 92 to a desired extent, e.g., as shown in FIG. 2B, the suture 30 may be secured, e.g., by tying one or more knots on the ends of the suture 30, e.g., to provide a continuous loop of suture 30 around the perimeter of the annular body 20. In addition or alternatively, one or more clips or other fasteners (not shown) may be used to secure the suture 30 relative to the constricted annular body 20. Any excess suture material may be cut or otherwise separated and removed, as desired. Optionally, the device 10 may be secured to adjacent tissue, e.g., by introducing one or more sutures through the fabric cover 40 and adjacent annulus tissue, e.g., around the perimeter of the device 10. The aorta 90 may then be closed and/or otherwise repaired using conventional methods.

Alternatively, with reference to FIG. 3, if an open or “C” shaped device 110 is provided, the device 110 may be positioned around the aortic root 92 without having to open the aorta 90, e.g., after completion of AV repair or VSARR, for example, if residual AR is detected in the patient using transesophageal echocardiography (“TEE”) or other imaging. Thus, in this alternative, the ends 112 of the body 120 may be separated and the device 110 positioned around a desired region, e.g., an enlarged region 92 similar to that shown in FIG. 2A. Once the device 110 is positioned, the ends 132 of the suture 130 may be pulled to tighten the device 110 around the region 92, e.g., with the hinge elements 126 compressing the region 92 substantially uniformly as the device 110 is tightened, similar to the enclosed device 10. For example, the ends 112 may initial abut one another once the device 110 is positioned around the region 92 and, during subsequent tightening, the features 124, 126 may deform to provide substantially symmetric radial compression of the region 92 being treated.

Alternatively, an annuloplasty device may be integrated into a tubular graft, e.g., permanently secured around one end of the graft (not shown). In this alternative, the graft may be used to replace an enlarged and/or otherwise damaged region of a valve annulus, e.g., to replace the enlarged region 92 of the aorta 90 shown in FIG. 2A immediately above the aortic valve 96. The desired length of the descending aorta may be severed and removed, and then the ends of the graft may be attached to the remaining ends, e.g., sutured using conventional methods. The annuloplasty device may then be positioned adjacent the native valve and, if desired, tightened to reduce regurgitation and/or otherwise support the valve annulus.

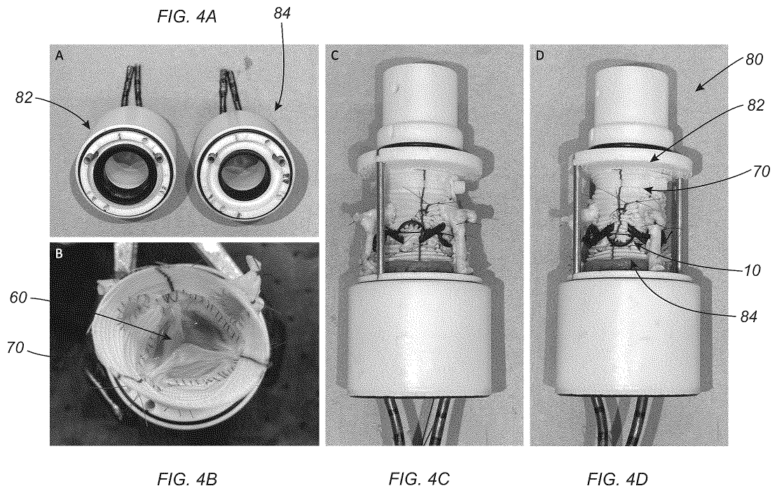

Turning to FIG. 4A-4D, an exemplary annuloplasty ring 10, such as the device 10 shown in FIGS. 1A-1D, is shown mounted to a model 80 of an aortic valve annulus. As shown, an excised aortic valve 60 was attached to a tubular graft 70, e.g., as shown in FIG. 4B, and opposite ends of the graft 70 were coupled to mounts 82, 84, with the upper mount 82 having a larger diameter than the lower mount 84, e.g., as shown in FIG. 4C. The annuloplasty ring was mounted around the graft 70 and tightened to constrict the graft 70, as shown in FIG. 4D.

For example, during experiments, porcine hearts were obtained and the aortic valve 60 was carefully explanted, preserving the left and right coronary arteries of at least 2 cm in length, 5 mm of the left ventricular outflow tract (LVOT), and aortic leaflets (not shown). To model residual AR from a dilated annulus after VSARR repair, each sample was first mounted to a dilated elastomeric sewing ring 82 with a diameter of 32 mm (FIG. 4A) using a continuous 4-0 polypropylene suture, and the coronary arteries were re-attached to the coronary cannulas using 6-0 polypropylene sutures.

Next, a VSARR procedure using a slightly over-sized Dacron graft was performed on the AV sample. Briefly, aortic wall tissue around the AV were scalloped and trimmed away, leaving 2 mm of aortic tissue distal to the AV leaflet attachment sites. Care was taken to avoid injuring the coronary artery ostium, and generous coronary buttons were created. A Dacron graft 70 was prepared with the annuloplasty device 10 placed around the proximal end. Next, the AV commissures were resuspended and attached to a size 32 straight Dacron graft using 4-0 polypropylene sutures. 2-0 polyester sutures were then used to anchor the aortic annulus to the proximal end of the Dacron graft in a scalloped fashion.

Next, 4-0 polypropylene sutures were used to create hemostatic suture lines attaching the aortic wall onto the Dacron graft. Lastly, using a high temperature electrocautery pen, two holes were generated in the Dacron graft right above the left and right coronary cusps at a level where the coronary buttons could be easily attached without torsion or stretching. Using 5-0 polypropylene sutures, the two coronary buttons were attached to the Dacron graft in a running fashion above the annuloplasty device (FIG. 2B). The distal Dacron graft was attached to the outflow mount, and the sample was used for data collection.

After the VSARR measurement collection, the annuloplasty device 10 was first attached to the Dacron graft 70 at the level of the aortic annulus using simple interrupted 2-0 silk sutures. Next, another 2-0 silk suture was placed through the channels of the device 10. This suture was tied down such that the Dacron graft diameter at the level of the annulus was reduced to 30 mm (FIG. 4D). The post ring annuloplasty repaired samples were then used for data collection.

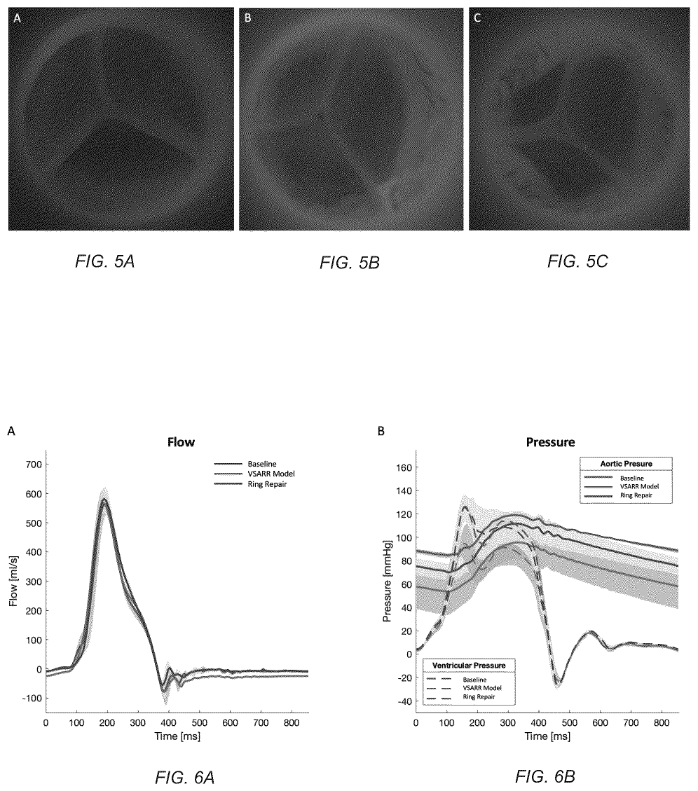

The VSARR model with aortic annular dilation successfully simulated residual AR. The en face view of an exemplary VSARR sample is shown in FIG. 5B. Compared to baseline where equal and adequate leaflet coaptations were observed (FIG. 5A), the VSARR model presented with a central regurgitation area due to the lack of leaflet coaptation. On average, leaflet coaptation height decreased from 1.1±0.1 cm, as measured from baseline samples, to 0.4±0.1 cm, as measured after the VSARR procedure (p=0.01). The mean systolic gradient across the AV was 4.3±1.3 mmHg at baseline and 4.8±1.5 mmHg after the VSARR procedure (p=0.72). Overall, the VSARR model consistently generated AR with an average regurgitant fraction of 23.6±6.9%, significantly increased from baseline 10.2±3.9% (p=0.03). Mean aortic flow tracings and pressure tracings are shown in FIGS. 6A and 6B. From the flow tracings, VSARR samples also demonstrated increased aortic regurgitation compared to baseline, as evidenced by the flow reversal during diastole. The mean arterial pressure and systolic pressure significantly decreased from 100.5±0.3 mmHg and 119.2±2.5 mmHg to 73.0±16.0 mmHg (p=0.04) and 95.7±14.3 mmHg (p=0.04), respectively.

The aortic ring annuloplasty repair using the device 10 successfully eliminated AR in the samples. As shown in the representative sample in FIG. 3C, leaflet coaptation plane was re-established and the central regurgitant area was eliminated using the device. The leaflet coaptation height after annuloplasty repair significantly increased from that measured in VSARR model to 0.9±0.1 cm (p=0.0004), a level similar to that measured in baseline (p=0.28). The mean systolic gradient across the AV after ring annuloplasty repair was 5.8±1.3 mmHg, similar to that of baseline (p=0.22) and post-VSARR (p=0.18). Overall, the EASE ring successfully reduced regurgitant fraction from the post-VSARR repair to 7.4±5.6% (p=0.05), which was similar to that measured from baseline (p=0.34). From the mean aortic flow tracings shown in FIG. 6A, the flow reversal during diastole disappeared after ring annuloplasty repair, and the overall flow tracing appeared similar to that obtained from the baseline measurements. The mean arterial pressure and systolic pressure both significantly increased from the post-VSARR repair to 89.8±8.7 mmHg (p=0.04) and 111.9±12.2 mmHg (p=0.02), similar to those obtained from baseline (p=0.09 and p=0.24), respectively.

As shown in FIG. 6A, mean aortic flow tracing confirmed the presence of aortic regurgitation in the VSARR model with flow reversal in diastole. Mean aortic flows were similar between the baseline and post-ring annuloplasty repair conditions. As shown in FIG. 6B, aortic and ventricular pressure tracings demonstrated lower pressures throughout the cardiac cycle in the VSARR model compared to baseline. After aortic ring annuloplasty repair, the pressures recovered to a level much closer to that obtained from baseline. Shaded regions represent standard deviation.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A prosthetic annuloplasty ring for constricting a valve annulus, comprising an annular body sized for positioning around an exterior of the valve annulus, the annular body comprising a compliant uni-body driven by a Bowden cable constriction mechanism to adjust an inner diameter of the device.

2. A prosthetic annuloplasty device for constricting a valve annulus, comprising:
an annular body defining a perimeter sized for positioning around an exterior of the valve annulus;
a plurality of channel elements on the annular body spaced apart from one another around the perimeter; and
a filament slidably received within channels in the channel elements and extending around the perimeter for adjusting an inner diameter of the annular body by tightening the filament.

3. The device of claim 2, wherein the annular body comprises a plurality of first elements and a plurality of second hinge elements alternating around the perimeter, the channel elements carried on the first elements.

4. The device of claim 3, wherein the channel elements are arranged on the respective first elements such that the channels are aligned generally circumferentially around the perimeter.

5. The device of claim 4, wherein the channels lie generally with a plane orthogonal to a central axis of the annular body.

6. The device of claim 3, wherein the first elements comprise one or more recesses or kerfing features located along an inner perimeter of the annular body.

7. The device of claim 6, wherein the one or more recesses or kerfing features accommodate bending of the first elements around the perimeter of the annular body.

8. The device of claim 3, wherein the hinge elements define a generally “C” or other arcuate shape with opposite ends that are coupled to the adjacent first elements.

9. The device of claim 8, wherein the hinge elements are configured such that, when the filament is tightened, an intermediate region of the hinge elements between the ends may bend or deform to direct the ends towards one another around the circumference, to reduce the diameter of the annular body.

10. The device of claim 8, wherein, the orientation of the hinge elements alternates around the perimeter of the annular body such that the ends of a first hinge element are located adjacent an upper end of the annular body and the intermediate region of the first hinge element is located adjacent a lower end of the annular body, and the ends of a second adjacent hinge elements are located adjacent the lower end and the intermediate region of the second adjacent hinge element is located adjacent the upper end.

11. A prosthetic annuloplasty device for constricting a valve annulus, comprising:
an open annular or “C shaped body defining a perimeter sized for positioning around an exterior of the valve annulus and including opposite ends;
a plurality of channel elements on the annular body spaced apart from one another around the perimeter; and
a filament slidably received within channels in the channel elements and extending around the perimeter for adjusting an inner diameter of the annular body by tightening the filament.

12. The device of claim 11, wherein the annular body comprises a plurality of first elements and a plurality of second hinge elements alternating around the perimeter, the channel elements carried on the first elements.

13. The device of claim 12, wherein the channel elements are arranged on the respective first elements such that the channels are aligned generally circumferentially around the perimeter.

14. The device of claim 13, wherein the channels lie generally with a plane orthogonal to a central axis of the annular body.

15. The device of claim 11, wherein the opposite ends are spaced apart from one another when the body is in a relaxed configuration.

16. The device of claim 15, wherein the body comprises flexible material such that a diameter of the body is reduced when the filament is tightened.

17. The device of claim 16, wherein the body is biased to the relaxed configuration.

18. The device of claim 11, wherein the body comprises flexible material such that the ends may be separated to open the body for positioning the body around a tubular structure.

19. The device of claim 18, wherein the body is biased to a relaxed state in which the ends are positioned adjacent one another when released after positioning the body around a tubular structure.

20-24. (canceled)

25. A method for constricting a valve annulus, comprising:
positioning an annular body externally around the valve annulus;
providing an elongate filament through a plurality of channels spaced apart from one another around a perimeter of the annular body; and
tightening the filament to reduce the perimeter of the annular body to constrict the valve annulus.

26-39. (canceled)
